(19) United States
(12) Patent Application Publication
(10) Pub. No.: US 2025/0034547 A1
(43) Pub. Date: Jan. 30, 2025

(54) COMPOSITIONS AND METHODS FOR THE PURIFICATION AND CONCENTRATION OF NUCLEIC ACIDS FROM LARGE-VOLUME SAMPLES

(71) Applicant: Promega Corporation, Madison, WI (US)

(72) Inventors: Subhanjan Mondal, Madison, WI (US); Zhiyang Zeng, Madison, WI (US); Kuei Hsuan Hsiao, Madison, WI (US); Kevin Kershner, Madison, WI (US); Wenhui Zhou, Madison, WI (US); James Cali, Madison, WI (US)

(21) Appl. No.: 18/783,882

(22) Filed: Jul. 25, 2024

Related U.S. Application Data
(60) Provisional application No. 63/515,410, filed on Jul. 25, 2023.

(51) Int. Cl. C12N 15/10; C12Q 1/6806

(52) U.S. Cl. CPC C12N 15/1013; C12Q 1/6806

(57) ABSTRACT

Provided herein are compositions and methods for purifying and concentrating nucleic acids from large-volume samples. In particular, reagents are provided for non-specifically binding total nucleic acid to a solid surface, separating bound nucleic acid from a large-volume sample, separating nucleic acid from amplification inhibitors, and/or eluting nucleic acids into a small volume amenable to further analysis.

### CROSS-REFERENCE TO RELATED APPLICATION

The present application claims priority to U.S. Provisional Patent Application Ser. No. 63/515,410 filed Jul. 25, 2023, which is hereby incorporated by reference in its entirety.

### FIELD

Provided herein are compositions and methods for purifying and concentrating nucleic acids from large-volume samples. In particular, reagents are provided for non-specifically binding total nucleic acid to a solid surface, separating bound nucleic acid from a large-volume sample, separating nucleic acid from amplification inhibitors, and/or eluting nucleic acids into a small volume amenable to further analysis.

### BACKGROUND

Purification of nucleic acid (e.g., total nucleic acid (TNA)) from large-volume sample (e.g., 10 ml-100 ml) that is amenable to high-throughput purification remains a challenge, particularly when samples are heterogenous environmental or biological samples that contain a high level of contaminants, some of which may act as PCR/RT-PCR inhibitors. In many testing cases (e.g., wastewater), nucleic acids, or entities containing the nucleic acids (e.g., cells, virus particles, etc.), are extremely diluted in test samples. In addition, these samples often contain many impurities (e.g., PCR inhibitors) that can interfere with downstream applications. Therefore, it is imperative to concentrate and purify nucleic acids from a large-volume sample (e.g., >10 ml). However, simple, efficient, and rapid concentration and purification of nucleic acids from samples of large volume remains challenging.

### SUMMARY

Provided herein are compositions and methods for purifying and concentrating nucleic acids from large-volume samples. In particular, reagents are provided for non-specifically binding total nucleic acid to a solid surface, separating bound nucleic acid from a large-volume sample, separating nucleic acid from amplification inhibitors, and/or eluting nucleic acids into a small volume amenable to further analysis.

In some embodiments, provided herein are methods of purifying nucleic acids from a large-volume sample comprising: (a) combining the large-volume sample with a first solid surface in the presence of a binding reagent, the binding reagent comprising divalent metal ions and a surfactant, and allowing cells, viral particles, and/or the nucleic acids in the large-volume sample to bind to the first solid surface; (b) separating the first solid surface from a liquid fraction of the large-volume sample; (c) contacting the first solid surface with a lysis/elution reagent comprising a chaotropic agent, anionic detergent, salt, reducing agent, and/or metal ion chelator, wherein cells and/or viral particles are lysed in the presence of the lysis/elution reagent and nucleic acids bound to the first solid surface and/or from within the cells and/or viral particles are eluted into the lysis/elution reagent; (d) combining the nucleic-acid-containing lysis/elution reagent with a second solid surface in the presence of a isopropanol and a pyrrolidone derivative to generate a purification mixture, allowing (i) nucleic acid from purification mixture to bind to the second solid surface and (ii) the pyrrolidone derivative to bind to amplification inhibitors present in the purification mixture; (e) separating the second solid surface from a liquid fraction of the purification mixture; and (f) contacting the second solid surface with an elution solution of 500 μl or less and allowing the nucleic acid from the purification mixture to elute off the solid surface and into the elution solution.

In some embodiments, the large-volume sample is a biological and/or environmental sample of 10 ml volume or greater (e.g., 10 ml, 15 ml, 20 ml, 30 ml, 40 ml, 50 ml, 75 ml, 100 ml, 150 ml 200 ml, 250 ml, or greater, or ranges therebetween). In some embodiments, the large-volume sample contains free nucleic acids, nucleic acid complexes with other biological macromolecules, cells, and/or viral particles. In some embodiments, the large-volume sample is wastewater.

In some embodiments, the first solid surface is a well, tube, bead, chip, plate, particle, membrane, or filter. In some embodiments, the first solid surface is a paramagnetic particle (PMP). In some embodiments, the PMP comprises a surface material capable of non-specifically binding to nucleic acids. In some embodiments, the surface material comprises silica, cellulose, chitosan, agarose, or Sepharose.

In some embodiments, the binding reagent comprises divalent metal ions selected from barium (Ba²⁺), copper [II] (Cu²⁺), calcium (Ca²⁺), magnesium (Mg²⁺), manganese [II] (Mn²⁺), zinc (Zn²⁺), iron [II] (Fe²⁺), nickel (Ni²⁺), cobalt (Co²⁺), tin [II] (Sn²⁺), cadmium (Cd²⁺), and lead [II] (Pb²⁺). In some embodiments, the binding reagent comprises Cu+, Zn²⁺, Co²⁺, Fe²⁺, and/or Ni²⁺. In some embodiments, the binding reagent comprises Zn²⁺. In some embodiments, non-hygroscopic salts are used, or excipients added to create a non-hygroscopic environment (FIGS. 5-6). An exemplary dry capture composition comprises: 120 mg Zinc Acetate dihydrate, 100 mg Cetylpyridinium chloride, 40 mg Dodecyltrimethylammonium bromide, and 25 mg of PMPs per capsule or tablet, although other formulations are described herein and within the scope herein.

In some embodiments, the binding reagent comprises a cationic surfactant. In some embodiments, the cationic surfactant is selected from behentrimonium chloride, benzalkonium chloride, benzethonium chloride, benzododecinium bromide, carbethopendecinium bromide, cetalkonium chloride, cetrimide, cetrimonium bromide, cetrimonium chloride, cetylpyridinium chloride, didecyldimethylammonium chloride, dimethyldioctadecylammonium bromide, dimethyldioctadecylammonium chloride, dioleoyl-3-trimethylammonium propane, domiphen bromide, lauryl methyl gluceth-10 hydroxypropyl dimonium chloride, octenidine dihydrochloride, olaflur, n-oleyl-1,3-propanediamine, pahutoxin, stearalkonium chloride, tetramethylammonium hydroxide, and thonzonium bromide.

In some embodiments, the binding reagent comprises a nonionic surfactant. In some embodiments, the nonionic surfactant is selected from alkyl polyglycoside, Cetomacrogol 1000, cetostearyl alcohol, cetyl alcohol, cocamide DEA, cocamide MEA, decyl glucoside, decyl polyglucose, glycerol monostearate, IGEPAL CA-630, isoceteth-20, Lauryl glucoside, Maltoside, monolaurin, mycosubtilin, narrow-range ethoxylate, nonidet P-40, nonoxynol-9, nonoxynols, NP-40, octaethylene glycol monododecyl ether, N-Octyl beta-D-thioglucopyranoside, octyl glucoside, oleyl alcohol, pentaethylene glycol monododecyl ether, polidocanol, Poloxamer, Poloxamer 407, polyethoxylated tallow amine, polyglycerol polyricinoleate, Polysorbate, Polysorbate 20, Polysorbate 80, Sorbitan, Sorbitan monolaurate, Sorbitan monostearate, Sorbitan tristearate, stearyl alcohol, Surfactin, Triton X-100, and Tween 80.

In some embodiments, the binding reagent is provided as a liquid reagent. In some embodiments, the first solid surface comprises beads and is provided within the liquid reagent as a suspension.

In some embodiments, the binding reagent is provided as a dry reagent. In some embodiments, the dry reagent is a powder, pellet, tablet, capsule, or disc. In some embodiments, the first solid surface comprises beads and is provided within the dry reagent. In some embodiments, combining the large-volume sample with the first solid surface in the presence of the binding reagent comprises dissolving the dry reagent in the large-volume sample.

In some embodiments, methods further comprise a wash step between steps (b) and (c) of washing the first solid surface with a wash solution that removes contaminants from first solid surface but allows the nucleic acids, cells, and/or viral particles to remain bound to the first solid surface.

In some embodiments, separating the first solid surface from the liquid fraction of the large-volume sample comprises mechanically or magnetically retaining the first solid surface while withdrawing the liquid fraction from the first solid surface by gravity, centrifugal force, or mechanical removal. In some embodiments, separating the first solid surface from the liquid fraction of the large-volume sample comprises removing mechanically, magnetically, or via centrifugal force the first solid surface from the liquid fraction.

In some embodiments, the lysis/elution reagent comprises a chaotropic agent. In some embodiments, the chaotropic agent is selected from sodium iodide, sodium perchlorate, guanidine thiocyanate, guanidine isothiocyanate, and guanidine hydrochloride

In some embodiments, the lysis/elution reagent comprises an anionic detergent. In some embodiments, the anionic detergent is selected from ammonium lauryl sulfate, sodium laureth sulfate, sodium lauryl sarcosinate, sodium myreth sulfate, sodium pareth sulfate, sodium stearte, sodium lauryl sulfate (sodium dodecyl sulfate (SDS)), a olefin sulfonate, and ammonium laureth sulfate.

In some embodiments, the lysis/elution reagent comprises a metal ion chelator. In some embodiments, the metal ion chelator is EDTA.

In some embodiments, the lysis/elution reagent comprises between 25 mM and 500 mM salt (e.g., 25 mM, 50 mM, 75 mM, 100 mM, 150 mM, 200 mM, 250 mM, 300 mM, 400 mM, 500 mM, or ranges therebetween). In some embodiments, the salt is selected from one or more of NaCl, KCl, MgCl₂, and (NH₄)₂SO₄.

In some embodiments, the lysis/elution reagent comprises a reducing agent. In some embodiments, the reducing agent is selected from dithiothreitol (DTT), 2-mercaptocthanol (BME), cysteamine, (2S)-2-amino-1,4-dimercaptobutane (DTBA), thiourea, 6-aza-2-thiothymine (ATT), and tris(2-carboxyethyl) phosphine (TCEP).

In some embodiments, the second solid surface is a well, tube, bead, chip, plate, particle, membrane, or filter. In some embodiments, the second solid surface is a paramagnetic particle (PMP). In some embodiments, the PMP comprises a surface material capable of non-specifically binding to nucleic acids. In some embodiments, the surface material comprises silica, cellulose, chitosan, agarose, or Sepharose.

In some embodiments, the pyrrolidone derivative is selected from polyvinylpolypyrrolidone (PVPP), poly(styrene-co-divinylbenzene, poly(1-vinylpyrrolidone-co-styrene), poly(1-vinylpyridine-co-styrene), and polyvinylpyrrolidone. In some embodiments, the pyrrolidone derivative is PVPP.

In some embodiments, methods further comprise a step of removing the pyrrolidone-derivative-bound amplification inhibitors from the purification mixture. In some embodiments, the pyrrolidone derivative is bound to particles to facilitate removal of the pyrrolidone-derivative-bound amplification inhibitors from the purification mixture. In some embodiments, the pyrrolidone derivative comprises PVPP-co-polystyrene beads.

In some embodiments, methods further comprise a step between steps (c) and (f) of washing the second solid surface with a wash solution that removes contaminants from second solid surface but allows the nucleic acids to remain bound to the second solid surface.

In some embodiments, provided herein are methods of purifying nucleic acids from a large-volume sample comprising: (a) combining the large-volume sample with a first set of paramagnetic particles (PMPs) having a surface material capable of non-specifically binding nucleic acids, cells, and viral particles in the presence of a binding reagent, the binding reagent comprising divalent metal ions and a surfactant, and allowing the nucleic acids, cells, and/or viral particles in the large-volume sample to bind to the PMPs; (b) separating the PMPs from a liquid fraction of the large-volume sample by applying a magnetic field to the PMPs and removing the PMPs from the liquid fraction or the liquid fraction from the PMPs; (c) contacting the PMPs with a lysis/elution reagent comprising a chaotropic agent, anionic detergent, salt, reducing agent, and/or metal ion chelator, wherein cells and/or viral particles are lysed in the presence of the lysis/elution reagent and nucleic acids bound to the PMPs and/or from within the cells and/or viral particles are eluted into the lysis/elution reagent; (d) combining the nucleic-acid-containing lysis/elution reagent with isopropanol and a pyrrolidone derivative and allowing the pyrrolidone derivative to bind to amplification inhibitors present in the purification mixture; (c) combining the purification mixture with a second set of PMPs having a surface material capable of non-specifically binding nucleic acids, and allowing nucleic acid from purification mixture to bind to the second set of PMPs; (e) separating the second set of PMPs from a liquid fraction of the purification mixture; and (f) contacting the second set of PMPs with an elution solution of 500 μl or less and allowing the nucleic acid from the purification mixture to elute off the second set of PMPs and into the elution solution.

In some embodiments, provided herein are nucleic acid capture compositions comprising: (a) paramagnetic particles (PMPs) having a surface material capable of non-specifically binding nucleic acids, cells, and viral particles; (b) divalent metal ions; and (c) a surfactant. In some embodiments, the surface material comprises silica, cellulose, chitosan, agarose, or Sepharose. In some embodiments, the divalent metal ions are selected from barium (Ba²⁺), copper [II] (Cu²⁺), calcium (Ca²⁺), magnesium (Mg²⁺), manganese [II] (Mn²⁺), zinc (Zn²⁺), iron [II] (Fe²⁺), nickel (Ni²⁺), cobalt (Co²⁺), tin [II] (Sn²⁺), cadmium (Cd²⁺), and lead [II] (Pb²⁺). In some embodiments, the divalent metal ions are selected from Cu+, Zn²⁺, Co²⁺, Fe²⁺, and/or Ni²⁺. In some embodiments, divalent metal ions comprise Zn²⁺. In some embodiments, the surfactant is a cationic surfactant. In some embodiments, the cationic surfactant is selected from behentrimonium chloride, benzalkonium chloride, benzethonium chloride, benzododecinium bromide, carbethopendecinium bromide, cetalkonium chloride, cetrimide, cetrimonium bromide, cetrimonium chloride, cetylpyridinium chloride, didecyldimethylammonium chloride, dimethyldioctadecylammonium bromide, dimethyldioctadecylammonium chloride, dioleoyl-3-trimethylammonium propane, domiphen bromide, lauryl methyl gluceth-10 hydroxypropyl dimonium chloride, octenidine dihydrochloride, olaflur, n-oleyl-1,3-propanediamine, pahutoxin, stearalkonium chloride, tetramethylammonium hydroxide, and thonzonium bromide. In some embodiments, the surfactant is a nonionic surfactant. In some embodiments, the nonionic surfactant is selected from alkyl polyglycoside, Cetomacrogol 1000, cetostearyl alcohol, cetyl alcohol, cocamide DEA, cocamide MEA, decyl glucoside, decyl polyglucose, glycerol monostearate, IGEPAL CA-630, isoceteth-20, Lauryl glucoside, Maltoside, monolaurin, mycosubtilin, narrow-range ethoxylate, nonidet P-40, nonoxynol-9, nonoxynols, NP-40, octaethylene glycol monododecyl ether, N-Octyl beta-D-thioglucopyranoside, octyl glucoside, oleyl alcohol, pentaethylene glycol monododecyl ether, polidocanol, Poloxamer, Poloxamer 407, polyethoxylated tallow amine, polyglycerol polyricinoleate, Polysorbate, Polysorbate 20, Polysorbate 80, Sorbitan, Sorbitan monolaurate, Sorbitan monostearate, Sorbitan tristearate, stearyl alcohol, Surfactin, Triton X-100, and Tween 80.

In some embodiments, capture compositions herein (e.g., comprising one or more of a solid surface (e.g., PMPs), surfactant, and divalent metal ions) can be deployed in any of multiple formats, for example as liquid solutions/reagents that are dispensed, as a solid form, such as a dissolvable capsule, as a pellet or tablet, or in a lyophilized form. The ability for large volume capture chemistry to be deployed in multiple formats offers great flexibility in the uses and applications of the technology. For example, one capsule or dry pellet (tablet) containing the capture composition is added to 40 ml of wastewater sample or to 10 ml of urine sample; once the capsule/pellet/tablet has dissolved, the capture composition is released into the sample. Often such samples are shipped to a testing lab, and the capture of the sample on the solid surface can happen during the transit of the sample to the testing lab. At the testing lab, the solid surface is separated and processed as described herein.

In some embodiments, the components of a nucleic acid capture composition herein are provided in a liquid suspension. In some embodiments, provided herein are kits comprising a container capable of securely containing a large-volume sample and a liquid suspension of a nucleic acid capture composition herein.

In some embodiments, the components of a nucleic acid capture composition herein are provided as a dry reagent. In some embodiments, the components of a nucleic acid capture composition herein are provided within a sealed capsule, wherein the capsule comprises a material that will dissolve when added to a liquid sample. In some embodiments, the components of a nucleic acid capture composition herein are provided as a lyophilized powder, pellet, tablet, or disc. Capsules can be made of any dissolvable polymer including, but not limited to, pullulan, methylcellulose, and gelatin. The components as solids are mixed or milled and dispensed within capsules. Tablets can be created by compaction of active ingredients and excipients (fillers, binders, glidants, and anti-adherents).

In some embodiments, provided herein are kits comprising a container having a nucleic acid capture composition (e.g., dry or liquid) contained therein and capable of securely containing a large-volume sample. In some embodiments, provided herein are methods comprising combining a large-volume sample with a nucleic acid capture composition herein. In some embodiments, provided herein are methods comprising: (a) providing a nucleic acid capture kit described herein, and (b) combining a large-volume sample and a nucleic acid capture composition described herein within the container. In some embodiments, the large-volume sample is an environmental sample. In some embodiments, the large-volume sample is a biological sample.

In some embodiments, provided herein are compositions (e.g., for binding and/or sequestering amplification inhibitors) comprising a pyrrolidone derivative and isopropanol. In some embodiments, a pyrrolidone derivative is present at about 0.005 g/ml to 0.5 g/ml (e.g., 0.005 g/ml, 0.01 g/ml, 0.02 g/ml, 0.05 g/ml, 0.1 g/ml, 0.2 g/ml, 0.5 g/ml, or any ranges in between and isopropanol or other alcohol present at concentrations between 30% to 100% or any ranges in between. In some embodiments, the pyrrolidone derivative is polyvinylpolypyrrolidone (PVPP). In some embodiments, the pyrrolidone derivative is PVPP-co-polystyrene. In some embodiments, provided herein are methods of removing or sequestering amplification inhibitors in a sample comprising contacting the sample with a pyrrolidone derivative composition described herein and allowing the pyrrolidone derivative to bind to the amplification inhibitors. In some embodiments, methods further comprise removing the pyrrolidone-derivative-bound amplification inhibitors from the sample. In some embodiments the pyrrolidone derivative is contained on an insoluble matrix or as an insoluble particle of size between 0.1 micron to 30 micron or is a soluble pyrrolidone derivative.

In some embodiments, provided herein are method: of preparing a large-volume sample for nucleic acid concentration comprising combining the large-volume sample with a solid surface in the presence of a binding reagent, the binding reagent comprising divalent metal ions and a surfactant, and allowing the nucleic acids, cells, and/or viral particles in the large-volume sample to bind to the solid surface. In some embodiments, the large-volume sample is a biological and/or environmental sample of 5 ml volume or greater (e.g., 5 ml, 10 ml, 15 ml, 20 ml, 30 ml, 40 ml, 50 ml, 75 ml, 100 ml, 150 ml 200 ml, 250 ml, or greater, or ranges therebetween). In some embodiments, the large-volume sample contains free nucleic acids, nucleic acid complexes with other biological macromolecules, cells, and/or viral particles. In some embodiments, the solid surface is a well, tube, bead, chip, plate, particle, membrane, or filter. In some embodiments, the solid surface is a paramagnetic particle (PMP). In some embodiments, the PMP comprises a surface material capable of non-specifically binding to nucleic acids. In some embodiments, the surface material comprises silica, cellulose, chitosan, agarose, or Sepharose. In some embodiments, the binding reagent comprises divalent metal ions selected from barium (Ba²⁺), copper [II] (Cu²⁺), calcium (Ca²⁺), magnesium (Mg²⁺), manganese [II] (Mn²⁺), zinc (Zn²⁺), iron [II] (Fe²⁺), nickel (Ni²⁺), cobalt (Co²⁺), tin [II] (Sn²⁺), cadmium (Cd²⁺), and lead [II] (Pb²⁺). In some embodiments, the binding reagent comprises Cu+, Zn²⁺, Co²⁺, Fe²⁺, and/or Ni²⁺. In some embodiments, the binding reagent comprises Zn²⁺. In some embodiments, the binding reagent comprises a cationic surfactant. In some embodiments, the cationic surfactant is selected from behentrimonium chloride, benzalkonium chloride, benzethonium chloride, benzododecinium bromide, carbethopendecinium bromide, cetalkonium chloride, cetrimide, cetrimonium bromide, cetrimonium chloride, cetylpyridinium chloride, didecyldimethylammonium chloride, dimethyldioctadecylammonium bromide, dimethyldioctadecylammonium chloride, dioleoyl-3-trimethylammonium propane, domiphen bromide, lauryl methyl gluceth-10 hydroxypropyl dimonium chloride, octenidine dihydrochloride, olaflur, n-oleyl-1,3-propanediamine, pahutoxin, stearalkonium chloride, tetramethylammonium hydroxide, and thonzonium bromide. In some embodiments, the binding reagent comprises a nonionic surfactant. In some embodiments, the nonionic surfactant is selected from alkyl polyglycoside, Cetomacrogol 1000, cetostearyl alcohol, cetyl alcohol, cocamide DEA, cocamide MEA, decyl glucoside, decyl polyglucose, glycerol monostearate, IGEPAL CA-630, isoceteth-20, Lauryl glucoside, Maltoside, monolaurin, mycosubtilin, narrow-range ethoxylate, nonidet P-40, nonoxynol-9, nonoxynols, NP-40, octaethylene glycol monododecyl ether, N-Octyl beta-D-thioglucopyranoside, octyl glucoside, oleyl alcohol, pentaethylene glycol monododecyl ether, polidocanol, Poloxamer, Poloxamer 407, polyethoxylated tallow amine, polyglycerol polyricinoleate, Polysorbate, Polysorbate 20, Polysorbate 80, Sorbitan, Sorbitan monolaurate, Sorbitan monostearate, Sorbitan tristearate, stearyl alcohol, Surfactin, Triton X-100, and Tween 80.

### BRIEF DESCRIPTION OF THE DRAWINGS

FIG. 1A-D. Divalent cations enhance the capture of various nucleic acids in a large-volume wastewater sample: (a) SARS-CoV-2 RNA, (b) Pepper mild mottle virus (PMMoV) RNA, (c) CrAssPhage DNA, and (d) Bacteroides HF183 DNA.

FIG. 2A-B. Effect of particle size and surface functionalization on nucleic acid extraction yield and purity: (a) PMMOV RNA and (b) SARS-CoV-2 RNA.

FIG. 3. Effect of various additives in binding buffer on nucleic acid capture.

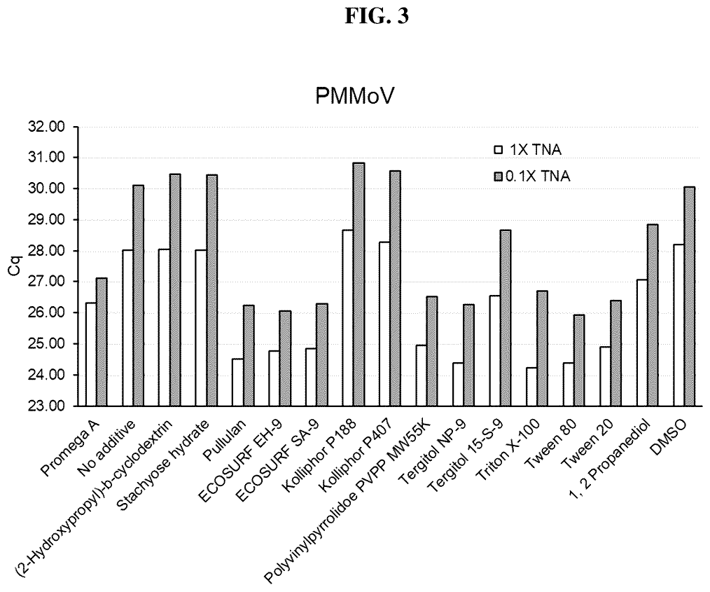

FIG. 4. Effect of various pyrrolidone derivatives on PCR efficiency.

FIG. 5. 40 ml wastewater sample was used to extract nucleic acid from using a two-component liquid format (reagent 1: paramagnetic particles in solution, and reagent 2: binding buffer containing: cationic surfactant and divalent metal ion), and a solid format containing paramagnetic particles, divalent metal ions, and surfactants enclosed in a pullulan capsule. The sample is contacted with reagents for these two formats for 30 minutes, and the paramagnetic particles separated using a magnetic stand. The paramagnetic particles are contacted with a lysis agent to elute the captured nucleic acid into the solution. The sample is clarified by placing on the magnetic stand again, and the eluted lysate is used to extract nucleic acid using the Maxwell® automated nucleic acid extractor. Purified nucleic acid is used to analyze microbial targets. Results indicate both liquid format and solid format have similar performance.

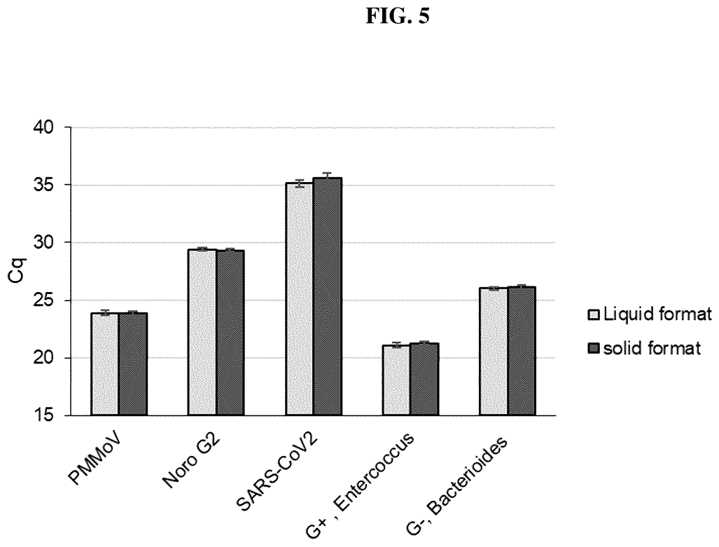

FIG. 6. Dried format containing cationic detergents and paramagnetic particles, but differing in the nature of the divalent metal ion. Zinc chloride (ZnCl₂) and Zinc sulphate heptahydrate (ZnSO₄·7H₂O) are hygroscopic and resulted in sogginess of the capsule. Zinc acetate dihydrate was not hygroscopic. Nucleic acid purified using any of the zinc salts resulted in similar yields.

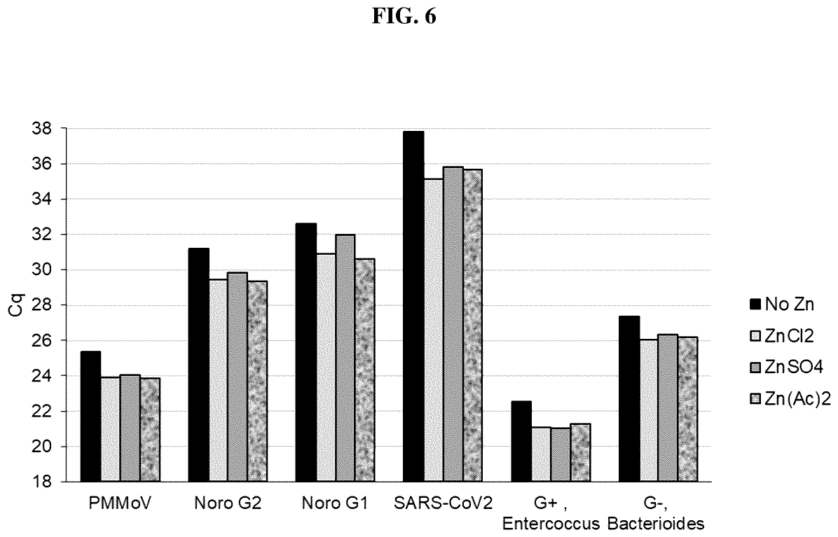

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a,” “an,” and “the” include plural reference unless the context clearly dictates otherwise.

As used herein, the term “comprise” and linguistic variations thereof denote the presence of recited feature(s), element(s), method step(s), etc., without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc., and any additional feature(s), element(s), method step(s), etc., that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “substantially” means that the recited characteristic, parameter, and/or value need not be achieved exactly, but that deviations or variations, including for example, tolerances, measurement error, measurement accuracy limitations and other factors known to skill in the art, may occur in amounts that do not preclude the effect the characteristic was intended to provide. A characteristic or feature that is substantially absent (e.g., a contaminant) may be one that is within the noise, beneath background, below the detection capabilities of the assay being used, or a small fraction (e.g., <1%, <0.1%, <0.01%, <0.001%, <0.00001%, <0.000001%, <0.0000001%) of the significant characteristic.

As used herein, the term “physiological conditions” encompasses any conditions compatible with living cells, e.g., predominantly aqueous conditions of a temperature, pH, salinity, chemical makeup, etc., that are compatible with living cells.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum, and the like. Sample may also refer to cell lysates or purified forms of the enzymes, peptides, and/or polypeptides described herein. Cell lysates may include cells that have been lysed with a lysing agent or lysates such as rabbit reticulocyte or wheat germ lysates. Sample may also include cell-free expression systems. Environmental samples include environmental material such as surface matter, soil, water, crystals, wastewater, and industrial samples. Such examples are not however to be construed as limiting the sample types applicable to the present invention.

The term “large-volume sample” refers to a sample of 5 ml or larger (e.g., 5 ml, 6 ml, 7 ml, 8 ml, 9 ml, 10 ml, 15 ml, 20 ml, 30 ml, 40 ml, 50 ml, 60 ml, 70 ml, 80 ml, 90 ml, 100 ml, or greater, or ranges therebetween).

### DETAILED DESCRIPTION

Provided herein are compositions and methods for purifying and concentrating nucleic acids from large-volume samples. In particular, reagents are provided for non-specifically binding total nucleic acid to a solid surface, separating bound nucleic acid from a large-volume sample, separating nucleic acid from amplification inhibitors, and/or eluting nucleic acids into a small volume amenable to further analysis.

In some embodiments, provided herein are compositions and methods for concentration and/or purification of nucleic acids from dilute and large-volume samples rapidly and efficiently. In some embodiments, the compositions and methods herein provide for concentration of nucleic acids from a large volume (e.g., >5 ml) into a small volume (e.g., <100 μl), liberation of nucleic acids from cells or viral particles, isolation of nucleic acids from sample contaminants, and/or removal of amplification inhibitors (e.g., present in the sample, introduced during the methods herein).

In some embodiments, the methods herein (or portions thereof) are performed manually (e.g., without automation) by being amenable to performing without expensive or complicated instrumentation, the methods herein (or various steps thereof) can be performed in the field and/or by a user without advanced science or laboratory training. In some embodiments, the methods herein (or various steps thereof) are performed using an automated instrument, such as an automated liquid handler (e.g., Promega Maxwell System) to reduce overall processing time and make the process semi-automated.

In some embodiments, provided herein are reagents that facilitate the binding (e.g., non-specific binding) of nucleic acids (e.g., DNA, RNA, single stranded, double stranded, TNA, etc.)

to a solid surface.

In some embodiments, a reagent to facilitate binding a nucleic acid in a large-volume sample comprises one or more types of cations. In some embodiments, the cations are monovalent, divalent, or trivalent. In some embodiments, a reagent to facilitate binding a nucleic acid in a large-volume sample comprises one or more types of divalent cations. In some embodiments, the divalent cations in such a reagent are selected from barium (Ba²⁺), copper [II] (Cu²⁺), calcium (Ca²⁺), magnesium (Mg²⁺), manganese [II] (Mn²⁺), zinc (Zn²⁺), iron [II] (Fe²⁺), nickel (Ni²⁺), cobalt (Co²⁺), tin [II] (Sn²⁺), cadmium (Cd²⁺), and lead [II] (Pb²⁺). In some embodiments, the divalent cation is provided as a salt with two monovalent anions (e.g., fluoride (F⁻), chloride (Cl⁻), bromide (Br⁻), iodide (I⁻), etc.) or one divalent anion (e.g., carbonate (CO₃²⁻), chromate (CrO₄²⁻), dichromate (Cr₂O₇²⁻), manganate (MnO₄²⁻), tetrathionate (S₄O₆²⁻), sulphide (S²⁻), sulphite (SO₃²⁻), sulphate (SO₄²⁻), oxide (O²⁻), zincate (ZnO₂²⁻), and thiosulphate (S₂O₃²⁻), etc.). An exemplary salt with divalent cations that finds use in reagents herein is ZnCl₂. In some embodiments, a divalent cation is present at about 0.1 mM to 200 mM (e.g., 0.1 mM, 0.2 mM, 0.5 mM, 1 mM, 5 mM, 10 mM, 20 mM, 50 mM, 100 mM, 200 mM, or any ranges therebetween)

In some embodiments, a reagent to facilitate binding of a nucleic acid in a large-volume sample comprises one or more types of polymers and/or surfactants.

In some embodiments, a reagent to facilitate binding a nucleic acid in a large-volume sample comprises a detergent or surfactant. In some embodiments, a detergent or surfactant is present at about 0.01 mol % to 5 mol % (e.g., 0.01%, 0.02%, 0.05%, 0.1%, 0.2%, 0.5%, 1%, 2%, 5%, 10%, or any ranges therebetween (e.g., 0.1 to 0.5%).

In some embodiments, a reagent to facilitate binding a nucleic acid in a large-volume sample comprises a polymer.

In some embodiments, the polymer is a naturally-occurring biopolymer. In some embodiments, the naturally-occurring biopolymer is selected from pullulan, trehalose, maltose, cellulose, dextran, and a combination of any thereof.

In some embodiments, the polymer is a cyclic saccharide polymer or a derivative thereof. In some embodiments, the polymer is hydroxypropyl β-cyclodextrin.

In some embodiments, the polymer is a synthetic polymer. In some embodiments, the synthetic polymer is selected from polystyrene, poly(meth) acrylate, and a combination of any thereof. In some embodiments, the synthetic polymer is a block copolymer comprising at least one poly(propylene oxide) block and at least one poly(ethylene oxide) block. In some embodiments, the synthetic polymer is a poloxamer. In some embodiments, the poloxamer is selected from poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407. In some embodiments, the poloxamer is poloxamer 407.

In some embodiments, suitable polymers for inclusion in the nucleic acid binding reagents herein are, for example, polyvinyl pyrrolidone (PVP), polyvinylpolypyrrolidone (PVPP), polyvinyl alcohol (PVA), polyvinyl alcohol low peroxide (PVA-LP), hydroxylpropyl methyl cellulose (HPMC), hydroxylpropyl cellulose (HPC), methyl cellulose, methacrylate polymers, cyclodextrin, dextrin, dextran, polyacrylic acid, chitosan, guar gum, xanthan gum, polyethylene oxide (e.g., polyethylene polypropylene oxide), poly(sodium vinylsulfonate), polyethylene glycol, poly(arginine), poly carbophil, polyvinyl pyrrolidone-co-vinyl acetate, a poloxamer (e.g., Pluronic® products available from BASF), alginate, trehalose, sucrose, inulin, or a combination or mixture thereof. In some embodiments, the composition comprises a polymer selected from PVP, PVA, methacrylate polymers, cyclodextrin, dextran, polyacrylic acid, chitosan, guar gum, xanthan gum, polyethylene oxide, polyethylene glycol, poly(arginine), poly carbophil, polyvinyl pyrrolidone-co-vinyl acetate, a poloxamer, or a combination or mixture thereof. In some embodiments, the composition comprises PVP, PVA, polyethylene oxide, or a mixture thereof. In some embodiments, the composition comprises PVP, PVA, or a mixture thereof. In some embodiments, the composition comprises PVP. In some embodiments, the composition comprises PVA.

In some embodiments, a surfactant is selected from polysorbate 20, polysorbate 40, and polysorbate 80.

In some embodiments, a reagent to facilitate binding of a nucleic acid in a large-volume sample comprises a cationic surfactant. Suitable cationic surfactants include, but are not limited to, behentrimonium chloride, benzalkonium chloride, benzethonium chloride, benzododecinium bromide, carbethopendecinium bromide, cetalkonium chloride, cetrimide, cetrimonium bromide, cetrimonium chloride, cetylpyridinium chloride, didecyldimethylammonium chloride, dimethyldioctadecylammonium bromide, dimethyldioctadecylammonium chloride, dioleoyl-3-trimethylammonium propane, domiphen bromide, lauryl methyl gluceth-10 hydroxypropyl dimonium chloride, octenidine dihydrochloride, olaflur, n-oleyl-1,3-propanediamine, pahutoxin, stearalkonium chloride, tetramethylammonium hydroxide, and thonzonium bromide.

In some embodiments, a reagent to facilitate binding of a nucleic acid in a large-volume sample comprises a non-ionic surfactant. Suitable non-ionic surfactants include, but are not limited to, alkyl polyglycoside, Cetomacrogol 1000, cetostearyl alcohol, cetyl alcohol, cocamide DEA, cocamide MEA, decyl glucoside, decyl polyglucose, glycerol monostearate, IGEPAL CA-630, isoceteth-20, Lauryl glucoside, Maltoside, monolaurin, mycosubtilin, narrow-range ethoxylate, nonidet P-40, nonoxynol-9, nonoxynols, NP-40, octaethylene glycol monododecyl ether, N-Octyl beta-D-thioglucopyranoside, octyl glucoside, oleyl alcohol, pentaethylene glycol monododecyl ether, polidocanol, Poloxamer, Poloxamer 407, polyethoxylated tallow amine, polyglycerol polyricinoleate, Polysorbate, Polysorbate 20, Polysorbate 80, Sorbitan, Sorbitan monolaurate, Sorbitan monostearate, Sorbitan tristearate, stearyl alcohol, Surfactin, Triton X-100, and Tween 80.

A binding reagent may also comprise additional components. Additional components may comprise a buffer, such as 2-morpholinoethanesulfonic acid monohydrate (MES), cacodylic acid, H₂CO₃/3, citric acid, bis(2-hydroxyethyl)-imino-tris-(hydroxymethyl)-methane (Bis-Tris), N-carbamoylmethylimidino acetic acid (ADA), 3-bis [tris(hydroxymethyl)methylamino]propane (Bis-Tris Propane), piperazine-1,4-bis(2-ethanesulfonic acid) (PIPES), N-(2-Acetamido)-2-aminoethanesulfonic acid (ACES), imidazole, N,N-bis(2-hydroxyethyl)-2-aminoethanesulfonic acid (BES), 3-(N-morpholino) propanesulphonic acid (MOPS), NaH₂PO₄/Na₂HPO₄, N-tris(hydroxymethyl)methyl-2-aminoethanesulfonic acid (TES), N-(2-hydroxyethyl)-piperazine-N′-2-ethanesulfonic acid (HEPES), N-(2-hydroxyethyl) piperazine-N′-(2-hydroxypropanesulfonic acid) (HEPPSO), triethanolamine, N-[tris(hydroxymethyl)methyl]glycine (Tricine), tris hydroxymethylaminoethane (Tris), glycineamide, N,N-bis(2-hydroxyethyl)glycine (Bicine), glycylglycine, N-tris(hydroxymethyl)methyl-3-aminopropanesulfonic acid (TAPS), or a combination thereof.

In some embodiments, provided herein are solid surfaces, substrates, matrices or supports that are capable of binding nucleic acids in a sequence agnostic manner. In some embodiments, a suitable material for the solid surface is selected that binds to total nucleic acids (e.g., DNA and RNA), long (e.g., mRNA, genomic DNA, viral RNA/DNA, etc.) and short (e.g., miRNA, siRNA, etc.) in the presence of the binding reagents herein in a sequence non-specific manner.

Suitable solid surfaces include, but are not limited to beads (e.g., magnetic beads), chips, tubes, plates, wells, particles, membranes, paper, etc. Surfaces may be porous. Surface may be spherical, flat, amorphous, or of any suitable shape. In some embodiments, the solid surface is a bead (or particle). Exemplary beads include magnetic beads, polymeric beads, and resin beads.

In some embodiments, a solid surface comprises a surface material capable of non-specifically binding (e.g., sequence agnostic) to nucleic acids such as silica, cellulose, chitosan, agarose, Sepharose. In some embodiments, a material herein (e.g., silica, cellulose, etc.) may be coupled to a synthetic or to a natural polymer, such as a polysaccharide grafted material (e.g., silica, cellulose, etc.), polyvinylpyrrolidone grafted material (e.g., silica, cellulose, etc.), polyethylene oxide grafted material (e.g., silica, cellulose, etc.), poly(2-hydroxyethylaspartamide) material (e.g., silica, cellulose, etc.), and poly(N-isopropylacrylamide) grafted material (e.g., silica, cellulose, etc.). In some embodiments, the material (e.g., silica, cellulose, etc.) may be functionalized or derivatized (e.g., conjugated to phosphate, sulfonate, amines, carboxylic acids, etc.).

Particular embodiments may utilize silica particles or silica-coated particles as the solid surface. Other embodiments herein utilize cellulose particles or cellulose-coated particles as the solid surface. Some embodiments utilize Sepharose particles or Sepharose-coated particles as the solid surface. Some embodiments utilize agarose particles or agarose-coated particles as the solid surface. Some embodiments utilize chitosan particles or chitosan-coated particles as the solid surface. The silica, cellulose, Sepharose, chitosan, or agarose of such particles may be derivatized, coupled to an additional reagent, etc.

In some embodiments, the solid surface is a particle (e.g., magnetic silica or magnetic cellulose particles). In some embodiments, the particles that find use in embodiments herein are 0.1 to 20 microns in diameter (e.g., 0.1, 0.2, 0.5, 1, 2, 3, 4, 5, 6, 7, 8, 9, 10, 15, 20, or ranges therebetween).

In some embodiments, the solid surface may include a native polymer, such as cross-linked carbohydrate material, such as agarose, agar, cellulose, dextran, chitosan, konjac, carrageenan, gellan, alginate etc. In some embodiments, the solid surface comprises a synthetic polymer such as polyacrylamide, polymethacrylate, a copolymer of polysaccharide (e.g., dextran) and agarose (e.g., a polyacrylamide/agarose composite) or a polysaccharide and N,N′-methylenebisacrylamide.

In some embodiments, solid surfaces may comprise one or more suitable materials as a nucleic acid binder or as a base material (e.g., to which a nucleic acid binder is coated, conjugated, attached, etc.), such as: Ahlstrom CytoSep, Cellulose nitrate, Cellulose acetate, Cellulose (e.g., Whatman FTA-DMPK-A, B, and C cards; Whatman ET 3/Chr; Whatman protein saver 903 cards; Whatman Grade 1 filter paper; Whatman FTA Elute; Ahlstrom 226 specimen collection paper; etc.), Noviplex Plasma Prep Cards, Polypropylene membrane, PVDF, Nitrocellulose membrane (Millipore Nitrocellular Hi Flow Plus) Polytetrafluoroethylene film, Mixed cellulose esters, Glass fiber media (e.g., Whatman uniflter plates glass fiber filter membrane, Agilent dried matrix spotting cards, Ahlstrom grade 8950, etc.), Plastic (e.g., Polyester, Polypropylene, Polythersulfene, poly(methacrylate), Acrylic polymers, polytetrafluoreten, etc.), natural and synthetic polymers (e.g., mixture of polymers, co-block polymers, etc.), sugars (e.g., pullulan, trehalose, maltose, sucrose, cellulose, etc.), polyamides (e.g., natural (e.g., wool, silk, etc.), synthetic (e.g., aramids, nylon, etc.), etc.), metals (e.g., aluminum, cadmium, chromium, cobalt, copper, iron, manganese, nickel, platinum, palladium, rhodium, silver, gold, tin, titanium, tungsten, vanadium, zinc, etc.), alloys (e.g., alloys of aluminum (e.g., Al—Li, alumel, duralumin, magnox, zamak, etc.), alloys of iron (e.g., steel, stainless steel, surgical stainless steel, silicon steel, tool steel, cast iron, Spiegeleisen, etc.), alloys of cobalt (e.g., stellite, talonite, etc.), alloys of nickel (e.g., German silver, chromel, mu-metal, monel metal, nichrome, nicrosil, nisil, nitinol, etc.), alloys of copper (e.g., beryllium copper, billon, brass, bronze, phosphor bronze, constantan, cupronickel, bell metal, Devarda's alloy, gilding metal, nickel silver, nordic gold, prince's metal, tumbaga, etc.), alloys of silver (e.g., sterling silver, etc.), alloys of tin (e.g., Britannium, pewter, solder, etc.), alloys of gold (electrum, white gold, etc.), amalgam, etc.), ELISPot plates, Immunoassay plates, Tissue culture plates, etc.

In some embodiments, the solid surface comprises magnetic or paramagnetic beads or particles. Magnetic beads allow for separation of the beads and bead-bound nucleic acids from a supernatant liquid (e.g., large-volume sample) via the application of a magnetic field and the washing away of the supernatant liquid. In some embodiments, magnetic beads may be coated and/or present a surface of any suitable material described herein, i.e., a material capable of non-specifically binding nucleic acid (e.g., TNA).

In some embodiments, a binding reagent is provided to facilitate binding of nucleic acid in the large-volume sample to a solid surface. In some embodiments, a binding reagent is a liquid reagent. In some embodiments, a binding reagent is dry reagent (e.g., lyophilized) and is provided as a powder, tablet, capsule, disc, etc. In some embodiments, the binding reagent comprises divalent metal ions (e.g., provided as a salt or in solution) and a polymer and/or surfactant. In some embodiments, the binding reagent also comprises the solid surface, particularly in embodiments in which the solid surface is provided as beads or particles. In some embodiments, a scalable tube, vial, syringe, or other container is provided containing the divalent metal ions, polymer and/or surfactant, and optionally beads/particles (e.g., as a liquid (e.g., suspension) or solid).

In some embodiments, methods of binding the nucleic acid from a large-volume sample are provided. In some embodiments, a large-volume sample comprising nucleic acids (e.g., free nucleic acids, nucleic acid complexes, cells, viruses, etc.) is contacted with a divalent metal ion, a polymer and/or surfactant, and a solid surface capable of non-specifically binding nucleic acid. In some embodiments, the divalent metal ion and polymer and/or surfactant are added to the sample as a single binding reagent and then the sample/binding reagent are contacted with the solid surface. In some embodiments, the divalent metal ion, polymer and/or surfactant, and solid surface (e.g., beads) are added to the sample. In some embodiments, the binding reagent, large-volume sample, and solid surface (if not included in the binding reagent) are combined under conditions that allow the nucleic acid in the sample to bind to the solid surface. Suitable conditions may include mixing, heating and/or cooling, providing sufficient time, etc.

In some embodiments, methods herein comprise a step of separating solid-surface-bond nucleic acid from the other components (e.g., liquid fraction) of the large-volume sample. In some embodiments, the solid surface (an bound nucleic acid) can be removed from the large-volume sample with minimal residual sample transferring with the solid surface. In some embodiments, the large-volume sample can be removed from the solid surface (an bound nucleic acid) remaining with the solid surface.

In some embodiments, the solid surface comprises a magnetic bead or particle (e.g., paramagnetic particles (PMPs)). In some embodiments, a magnetic force is applied to the sample containing the magnetic beads. In some embodiments, the magnetic field is used to remove the magnetic beads from the sample. In some embodiments, the magnetic field is used to hold the magnetic beads still (e.g., within a vessel) as the liquid of the large-volume sample is removed (e.g., from the vessel).

In some embodiments, wash solutions are provided herein for use in various steps. In some embodiments, a wash solution is applied to the solid surface (with nucleic acid bound thereto) following removal of the solid surface from the large-volume sample (or removal of the large-volume sample from the solid surface). In some embodiments, a wash solution comprises water. A wash solution may further comprise one or more salts, buffers, surfactants, polymers, etc., such as those that are described herein and/or understood in the field.

In some embodiments, provided herein is a lysis/elution reagent for removing nucleic acids from the solid surface. In some embodiments, nucleic acids are directly bound to the solid surface; in such cases, the lysis/elution reagent disrupts the interaction of the nucleic acids with the solid surface, thereby allowing the nucleic acid to be freed from the solid surface and released into the lysis/elution reagent. In some embodiments, nucleic acids contained within cell or viral particles, and the cells/viral particles are bound to the solid surface; in such cases, the lysis/elution reagent causes lysis of the cells/viral particles, liberation of the nucleic acids therefrom, and release of the nucleic acids into the lysis/elution reagent.

In some embodiments, the lysis/elution reagent comprises one or more salts. In some embodiments, the salt concentration in the lysis/elution reagent is between 25 and 500 mM (e.g., 25 mM, 50 mM, 75 mM, 100 mM, 150 mM, 250 mM, 300 mM, 400 mM, 500 mM, or ranges therebetween). Exemplary salts for inclusion in a lysis/elution reagent herein include, but are not limited to NaCl, KCl, MgCl₂, and (NH₄)₂SO₄.

In some embodiments, the lysis/elution reagent comprises one or more anionic surfactants. Examples of anionic surfactants that may find use in a lysis/elution reagent herein include but are not limited to ammonium lauryl sulfate, sodium laureth sulfate, sodium lauryl sarcosinate, sodium myreth sulfate, sodium pareth sulfate, sodium stearte, sodium lauryl sulfate (sodium dodecyl sulfate (SDS)), a olefin sulfonate, and ammonium laureth sulfate. In some embodiments, the lysis/elution reagent does not contain SDS. In some embodiments, an anionic surfactant is present at a concentration of 0.1-5% by weight (e.g., 0.1, 0.2, 0.5, 1, 2, 3, 4, 5, or ranges therebetween).

In some embodiments, the lysis/elution reagent comprises one or more chaotropic agents. Examples of chaotropic agents that may find use in a lysis/elution reagent herein include but are not limited to sodium iodide, urea, sodium perchlorate, guanidine thiocyanate, guanidine isothiocyanate and guanidine hydrochloride. In some embodiments, a chaotropic agent is present at a concentration of 0.1-7% by weight (e.g., 0.1, 0.2, 0.5, 1, 2, 3, 4, 5, 6, 7, or ranges therebetween).

In some embodiments, the lysis/elution reagent comprises a metal ion chelator, such as EDTA. In some embodiments, EDTA is present at a concentration of 1-200 mM (e.g., 1 mM, 2 mM, 5 mM, 10 mM, 20 mM, 50 mM, 100 mM, 200 mM, or ranges therebetween).

In some embodiments, the lysis/elution reagent comprises a reducing agent. Examples of reducing agent that may find use in a lysis/elution reagent herein include but are not limited to dithiothreitol (DTT), 2-mercaptocthanol (BME), cysteamine, (2S)-2-amino-1,4-dimercaptobutane (DTBA), thiourea, 6-aza-2-thiothymine (ATT), tris(2-carboxyethyl) phosphine (TCEP), or the like.

In some embodiments, the lysis/elution reagent comprises a buffer. Examples of buffers that may find use in a lysis/elution reagent herein include but are not limited to 2-morpholinoethanesulfonic acid monohydrate (MES), cacodylic acid, H₂CO₃/3, citric acid, bis(2-hydroxyethyl)-imino-tris-(hydroxymethyl)-methane (Bis-Tris), N-carbamoylmethylimidino acetic acid (ADA), 3-bis [tris(hydroxymethyl)methylamino]propane (Bis-Tris Propane), piperazine-1,4-bis(2-ethanesulfonic acid) (PIPES), N-(2-Acetamido)-2-aminoethanesulfonic acid (ACES), imidazole, N,N-bis(2-hydroxyethyl)-2-aminoethanesulfonic acid (BES), 3-(N-morpholino) propanesulphonic acid (MOPS), NaH₂PO₄/Na₂HPO₄, N-tris(hydroxymethyl)methyl-2-aminoethanesulfonic acid (TES), N-(2-hydroxyethyl)-piperazine-N′-2-ethanesulfonic acid (HEPES), N-(2-hydroxyethyl) piperazine-N′-(2-hydroxypropanesulfonic acid) (HEPPSO), triethanolamine, N-[tris(hydroxymethyl)methyl]glycine (Tricine), tris hydroxymethylaminoethane (Tris), glycineamide, N,N-bis(2-hydroxyethyl)glycine (Bicine), glycylglycine, N-tris(hydroxymethyl)methyl-3-aminopropanesulfonic acid (TAPS), or a combination thereof.

An exemplary compositions for a lysis/elution reagent (example components and concentrations) is 50 mM Tris-HCl (pH 7.5), 4.5 mM guanidine HCl, 50 mM EDTA, 1% SDS, and 6M urea.

In some embodiments, one or more of the steps in the purification/concentration methods described herein (e.g., binding to solid surface, lysis/elution from the solid surface, etc.) can introduce agents into the sample (e.g., the nucleic acid containing fraction) that can inhibit downstream analysis (e.g., inhibitors of nucleic acid amplification). Examples of such inhibitors include, but are not limited to excess salts (e.g., NaCl, KCl, etc.), surfactants, chaotropic agents, etc. In some embodiments, an agent is added to the sample (e.g., the nucleic acid containing fraction) after elution from the solid surface to bind and/or sequester inhibitors of nucleic acid amplification. In some embodiments, a pyrrolidone derivative is added to facilitate removal of amplification inhibitors. Suitable pyrrolidone derivatives may include polyvinylpolypyrrolidone (PVPP), poly(styrene-co-divinylbenzene, poly(1-vinylpyrrolidone-co-styrene), poly(1-vinylppyridine-co-styrene), polyvinylpyrrolidone, etc. In some embodiments, polyvinylpolypyrrolidone (PVPP) is added to remove inhibitors of amplification. In some embodiments, the reagent to facilitate removal of amplification inhibitors (e.g., PVPP or other pyrrolidone derivative) is added to a concentration of 0.1 to 500 μM (e.g., 0.1 μM, 0.2 μM, 0.5 μM, 1 μM, 2 μM, 5 μM, 10 μM, 20 μM, 50 μM, 100 μM, 200 μM, 500 μM, or ranges therebetween). In some embodiments, free PVPP is added to the eluted nucleic acid sample. In some embodiments, PVPP linked to a solid surface (e.g., PVPP-co-polystyrene beads) is added to the sample. The PVPP binds to the amplification inhibitors. In some embodiments, removal of the PVPP (e.g., PVPP-co-polystyrene beads) from the sample (e.g., by application of a magnetic field, by filtration, etc.) reduces the concentration of amplification inhibitors in the sample. In some embodiments, a pyrrolidone derivative other than PVPP is linked to a particle (polystyrene beads or other) and used for sequestration and/or removal of amplification inhibitors.

In some embodiments, amplification inhibitor removal is performed in the presence of an organic solvent (e.g., the pyrrolidone derivative and organic solvent are added to the post-lysis/eluted sample). In some embodiments, the organic solvent is isopropanol. In some embodiments, the organic solvent is a water-miscible solvent, such as acetone, acetic acid, butanediol, ethanol, ethylene glycol, methanol, isopropanol, etc.

In some embodiments, following elution of the nucleic acid from the solid surface and sequestering and/or removal of amplification inhibitors, the nucleic acid in the sample is contacted with a second solid surface. In particular embodiments, that second solid surface comprises bead with a surface material that non-specifically binds to nucleic acid (e.g., silica) as described herein. In some embodiments, the nucleic acids are bound to the second solid surface, and the solid-surface-bound nucleic acids are separated from the liquid portion of the sample (e.g., by mechanical, centrifugal, or magnetic force). In some embodiments, the solid-surface-bound nucleic acids are washed with water, a buffered aqueous solution, etc. In some embodiments, the nucleic acid is subsequently eluted from the second solid surface into a small volume (e.g., 10 μl, 20 μl, 30 μl, 40 μl, 50 μl, 75 μl, 100 μl, 125 μl, 150 μl, 200 μl, or ranges therebetween) elution solution (e.g., water).

In some embodiments, following purification of nucleic acid by the methods herein, the concentrated nucleic acid is subjected to one or more techniques for analysis. In particular embodiments, the nucleic acid is amplified during analysis or for subsequence analysis. The particular amplification technique utilized is selected by the skilled artisan based on the type of nucleic acid to be analyzed and the desired outcome of the analysis/amplification. In some embodiments, the analysis comprises detecting/quantifying the nucleic acid, detecting/quantifying a particular type or sequence of nucleic acid within the concentrated nucleic acid, sequencing the nucleic acid (or a portion thereof), etc. For example, in some embodiments, the disclosed methods further comprise a step of analyzing or processing the nucleic acid, e.g., using polymerase chain reaction (PCR), reverse transcription PCR (RT-PCR), quantitative PCR, quantitative reverse transcription PCR (RT-qPCR), real time PCR, hot start PCR, single cell PCR, nested PCR, in situ colony PCR, digital PCR (dPCR), Droplet Digital™ PCR (ddPCR), emulsion PCR, ligase chain reaction (LCR), transcription based amplification system (TAS), nucleic acid sequence-based amplification (NASBA), strand displacement amplification (SDA), rolling circle amplification (RCA), hyper-branched RCA (HRCA), isothermal amplification, gel electrophoresis, capillary electrophoresis, mass spectrometry, fluorescence detection, ultraviolet spectrometry, hybridization assays, DNA or RNA sequencing, reverse transcription, next generation sequencing (NGS), or the like.

### EXPERIMENTAL

### Example 1

### Effect of Metal Ions on Nucleic Acid Yields

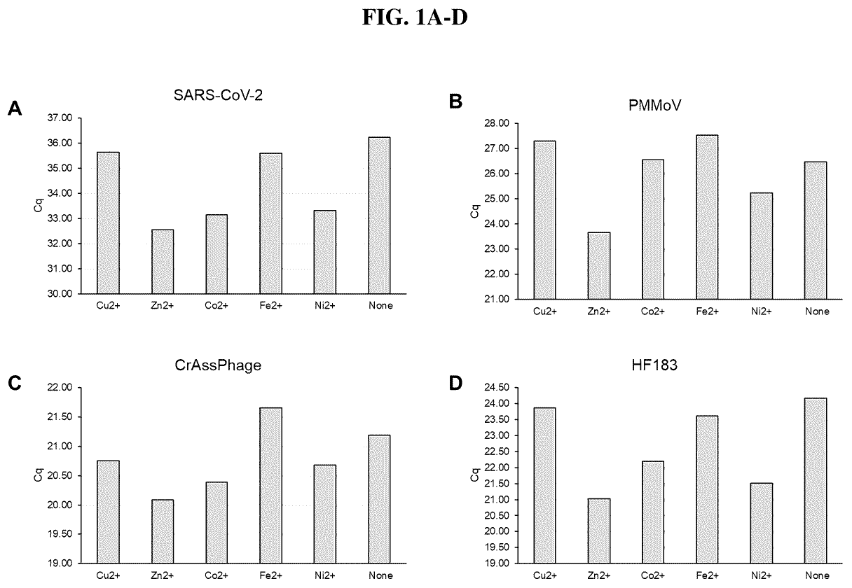

Experiments were conducted to determine the effect of divalent cations on capture of nucleic acids in a large-volume sample (FIG. 1). Magnetized silica beads in the presence of 10 mM of various metal ions (Cu²⁺, Zn²⁺, Co²⁺, Fe²⁺, Ni²⁺) or in the absence of any metal ion (MES Buffer, pH 6.0) were incubated with wastewater samples to capture biomass for 10 minutes. Captured biomass was separated using a magnetic stand and lysed using a buffer containing chaotropic agents, anionic detergents. Eluted lysate was used to purify nucleic acid using Maxwell automated purification system. The purified nucleic acid is used to analyze DNA (CrAssPhage a DNA virus and Bacteroides HF183, both human fecal marker with DNA genomes) and RNA (SARS-CoV-2 and Pepper mild mottle virus (PMMOV; a human fecal marker)) analytes in the sample.

### Example 2

### Effect of Particle Size and Surface Conjugation on Nucleic Acid Yields

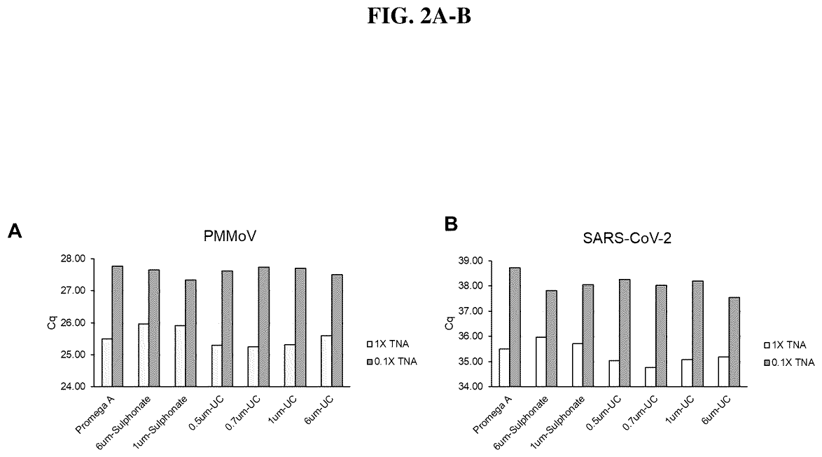

Experiments were conducted to determine the effect of particle size and surface conjugation in the presence of Zn2+ to capture biomass from environmental samples. Magnetic silica or cellulose particles of different sizes, 6, 1, 0.7, 0.5 microns, were either used unconjugated (UG) or conjugated with functional groups including, but not limited to, sulphonate and carboxylic acid and incubated with 40 ml of wastewater samples in the presence of Zn²⁺. The concentration of Zn²⁺ can be >0.001 mM. Captured biomass is separated using a magnetic stand and lysed using a buffer containing chaotropic agents and anionic detergents. Nucleic acid from the eluted lysate was purified using a Maxwell® automated purification system. The purified nucleic acid was used to analyze RNA (SARS-CoV-2 and Pepper mild mottle virus PMMoV (a human fecal marker)) targets in the sample (FIG. 2). Total Nucleic Acid (TNA) purified were also diluted 10-fold and analyzed for the same targets to determine the amount of inhibitors present in the eluted TNA. Results show smaller particles (silica or cellulose) are more efficient in capturing and eluting nucleic acid with greater yields and purity. Functionalization of silica or cellulose surfaces did not improve the capture, yields, or purity of the eluted TNA.

### Example 3

### Effect of Pyrrolidone Derivatives on PCR Efficiency

It is believed that anionic detergents and/or other reagents/contaminants from the various steps of the methods herein may compete with the charged groups on the silica/cellulosic surface and release all the captured material to the solution phase. The lysate containing the nucleic acid also contains contaminants that can be inhibitors of amplification reaction (e.g., reverse transcriptase and DNA polymerase inhibitors). Experiments were conducted during development of embodiments herein to identify reagents that could be included in steps of the methods herein to reduce or eliminate amplification inhibitors from the nucleic acid containing fraction.

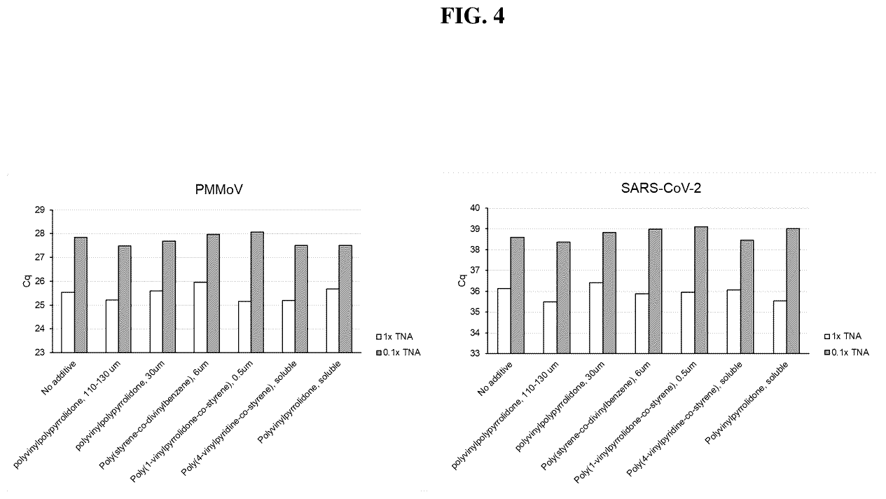

The Maxwell® automated system is a magnetic particle mover. The system includes a cartridge consisting of compartmentalized chambers for different reagent formulations. The eluted lysate was added to a well of the Maxwell® cartridge that contained isopropanol. The mixture of chaotropic agents and isopropanol creates an environment for attachment of nucleic acid with the magnetic resin therein. In the Maxwell® purification step, a fresh set of magnetic silica or cellulosic particles were used to extract the nucleic acid. Various insoluble or soluble pyrrolidone derivatives were included. Several polyvinylpyrrolidone derivatives helped eliminate PCR inhibitors as evident from a comparison of a 10-fold dilution (FIG. 4).

1. A method of purifying nucleic acids from a large-volume sample comprising:
(a) combining the large-volume sample with a first solid surface in the presence of a binding reagent, the binding reagent comprising divalent metal ions and a surfactant, and allowing cells, viral particles, and/or the nucleic acids in the large-volume sample to bind to the first solid surface;
(b) separating the first solid surface from a liquid fraction of the large-volume sample;
(c) contacting the first solid surface with a lysis/elution reagent comprising a chaotropic agent, anionic detergent, salt, reducing agent, and/or metal ion chelator, wherein cells and/or viral particles are lysed in the presence of the lysis/elution reagent and nucleic acids bound to the first solid surface and/or from within the cells and/or viral particles are eluted into the lysis/elution reagent;
(d) combining the nucleic-acid-containing lysis/elution reagent with a second solid surface in the presence of a isopropanol and a pyrrolidone derivative to generate a purification mixture, allowing (i) nucleic acid from the purification mixture to bind to the second solid surface and (ii) the pyrrolidone derivative to bind to amplification inhibitors present in the purification mixture;
(e) separating the second solid surface from a liquid fraction of the purification mixture; and
(f) contacting the second solid surface with an elution solution of 500 μl or less and allowing the nucleic acid from the purification mixture to elute off the solid surface and into the elution solution.

2. The method of claim 1, wherein the large-volume sample is a biological and/or environmental sample of 5 ml volume or greater.

3. The method of claim 1, wherein the large-volume sample contains free nucleic acids, nucleic acid complexes with other biological macromolecules, cells, and/or viral particles.

4. The method of claim 1, wherein the first solid surface is a well, tube, bead, chip, plate, particle, membrane, or filter.

5. The method of claim 4, wherein the first solid surface is a paramagnetic particle (PMP).

6. The method of claim 5, wherein the PMP comprises a surface material capable of non-specifically binding to cells, viruses, and/or nucleic acids.

7. The method of claim 6, wherein the surface material comprises silica, cellulose, chitosan, agarose, or Sepharose.

8. The method of claim 1, wherein the binding reagent comprises divalent metal ions selected from barium (Ba²⁺), copper [II] (Cu²⁺), calcium (Ca²⁺), magnesium (Mg²⁺), manganese [II] (Mn²⁺), zinc (Zn²⁺), iron [II] (Fe²⁺), nickel (Ni²⁺), cobalt (Co²⁺), tin [II] (Sn²⁺), cadmium (Cd²⁺), and lead [II] (Pb²⁺).

9. The method of claim 8, wherein the binding reagent comprises Cu+, Zn²⁺, Co²⁺, Fe²⁺, and/or Ni²⁺.

10. The method of claim 9, wherein the binding reagent comprises Zn²⁺.

11. The method of claim 1, wherein the binding reagent comprises a cationic surfactant.

12. The method of claim 11, wherein the cationic surfactant is selected from behentrimonium chloride, benzalkonium chloride, benzethonium chloride, benzododecinium bromide, carbethopendecinium bromide, cetalkonium chloride, cetrimide, cetrimonium bromide, cetrimonium chloride, cetylpyridinium chloride, didecyldimethylammonium chloride, dimethyldioctadecylammonium bromide, dimethyldioctadecylammonium chloride, dioleoyl-3-trimethylammonium propane, domiphen bromide, lauryl methyl gluceth-10 hydroxypropyl dimonium chloride, octenidine dihydrochloride, olaflur, n-oleyl-1,3-propanediamine, pahutoxin, stearalkonium chloride, tetramethylammonium hydroxide, and thonzonium bromide.

13. The method of claim 1, wherein the binding reagent comprises a nonionic surfactant.

14. The method of claim 13, wherein the nonionic surfactant is selected from alkyl polyglycoside, Cetomacrogol 1000, cetostearyl alcohol, cetyl alcohol, cocamide DEA, cocamide MEA, decyl glucoside, decyl polyglucose, glycerol monostearate, IGEPAL CA-630, isoceteth-20, Lauryl glucoside, Maltoside, monolaurin, mycosubtilin, narrow-range ethoxylate, nonidet P-40, nonoxynol-9, nonoxynols, NP-40, octaethylene glycol monododecyl ether, N-Octyl beta-D-thioglucopyranoside, octyl glucoside, oleyl alcohol, pentaethylene glycol monododecyl ether, polidocanol, Poloxamer, Poloxamer 407, polyethoxylated tallow amine, polyglycerol polyricinoleate, Polysorbate, Polysorbate 20, Polysorbate 80, Sorbitan, Sorbitan monolaurate, Sorbitan monostearate, Sorbitan tristearate, stearyl alcohol, Surfactin, Triton X-100, and Tween 80.

15. The method of claim 1, further comprising a step between steps (b) and (c) of washing the first solid surface with a wash solution that removes contaminants from first solid surface but allows the nucleic acids, cells, and/or viral particles to remain bound to the first solid surface.

16. The method of claim 1, wherein separating the first solid surface from the liquid fraction of the large-volume sample comprises mechanically or magnetically holding the first solid surface while withdrawing the liquid fraction from the first solid surface by gravity, centrifugal force, or mechanical removal.

17. The method of claim 1, wherein separating the first solid surface from the liquid fraction of the large-volume sample comprises removing mechanically, magnetically, or via centrifugal force the first solid surface from the liquid fraction.

18. The method of claim 1, wherein the lysis/elution reagent comprises a chaotropic agent.

19. The method of claim 18, wherein the chaotropic agent is selected from sodium iodide, sodium perchlorate, guanidine thiocyanate, guanidine isothiocyanate and guanidine hydrochloride

20. The method of claim 1, wherein the lysis/elution reagent comprises an anionic detergent.

21. The method of claim 20, wherein the anionic detergent is selected from ammonium lauryl sulfate, sodium laureth sulfate, sodium lauryl sarcosinate, sodium myreth sulfate, sodium pareth sulfate, sodium stearte, sodium lauryl sulfate (sodium dodecyl sulfate (SDS)), a olefin sulfonate, and ammonium laureth sulfate.

22. The method of claim 1, wherein the lysis/elution reagent comprises a metal ion chelator.

23. The method of claim 22, wherein the metal ion chelator is EDTA.

24. The method of claim 1, wherein the lysis/elution reagent comprises a metal ion chelator.

25. The method of claim 24, wherein the metal ion chelator is EDTA.

26. The method of claim 1, wherein the lysis/elution reagent comprises between 10 mM and 500 mM salt.

27. The method of claim 26, wherein the salt is selected from one or more of NaCl, KCl, MgCl₂, and (NH₄)₂SO₄.

28. The method of claim 1, wherein the lysis/elution reagent comprises a reducing agent.

29. The method of claim 28, wherein the reducing agent is selected from dithiothreitol (DTT), 2-mercaptoethanol (BME), cysteamine, (2S)-2-amino-1,4-dimercaptobutane (DTBA), thiourea, 6-aza-2-thiothymine (ATT), and tris)2-carboxyethyl) phosphine (TCEP).

30. The method of claim 1, wherein the second solid surface is a well, tube, bead, chip, plate, particle, membrane, or filter.

31. The method of claim 30, wherein the second solid surface is a paramagnetic particle (PMP).

32. The method of claim 31, wherein the PMP comprises a surface material capable of non-specifically binding to nucleic acids.

33. The method of claim 32, wherein the surface material comprises silica, cellulose, chitosan, agarose, or Sepharose.

34. The method of claim 1, wherein the pyrrolidone derivative is selected from polyvinylpolypyrrolidone (PVPP), poly(styrene-co-divinylbenzene, poly(1-vinylpyrrolidone-co-styrene), poly(1-vinylppyridine-co-styrene), and polyvinylpyrrolidone.

35. The method of claim 34, wherein the pyrrolidone derivative is PVPP.

36. The method of claim 1, further comprising a step of removing the pyrrolidone-derivative-bound amplification inhibitors from the purification mixture.

37. The method of claim 36, wherein the pyrrolidone derivative is bound to a particles to facilitate removal of the pyrrolidone-derivative-bound amplification inhibitors from the purification mixture.

37. The method of claim 37 wherein the pyrrolidone derivative comprises PVPP-co-polystyrene beads.

38. The method of claim 1, further comprising a step between steps (e) and (f) of washing the second solid surface with a wash solution that removes contaminants from second solid surface but allows the nucleic acids to remain bound to the second solid surface.

39. The method of claim 1, wherein the binding reagent is provided as a liquid reagent.

40. The method of claim 39, wherein the first solid surface comprises beads and is provided within the liquid reagent as a suspension.

39. The method of claim 1, wherein the binding reagent is provided as a dry reagent.

40. The method of claim 39, wherein the dry reagent is a powder, pellet, tablet, capsule, or disc.

41. The method of claim 40, wherein the first solid surface comprises beads and is provided within the dry reagent.

42. The method of one of claims 39-41, wherein combining the large-volume sample with the first solid surface in the presence of the binding reagent comprises dissolving the dry reagent in the large-volume sample.

43. A method of purifying nucleic acids from a large-volume sample comprising:
(a) combining the large-volume sample with a first set of paramagnetic particles (PMPs) having a surface material capable of non-specifically binding nucleic acids, cells, and viral particles in the presence of a binding reagent, the binding reagent comprising divalent metal ions and a surfactant, and allowing cells, viral particles, and/or the nucleic acids in the large-volume sample to bind to the PMPs;
(b) separating the PMPs from a liquid fraction of the large-volume sample by applying a magnetic field to the PMPs and removing the PMPs from the liquid fraction or the liquid fraction from the PMPs;
(c) contacting the PMPs with a lysis/elution reagent comprising a chaotropic agent, anionic detergent, salt, reducing agent, and/or metal ion chelator, wherein cells and/or viral particles are lysed in the presence of the lysis/elution reagent and nucleic acids bound to the PMPs and/or from within the cells and/or viral particles are eluted into the lysis/elution reagent;
(d) combining the nucleic-acid-containing lysis/elution reagent with isopropanol and a pyrrolidone derivative and allowing the pyrrolidone derivative to bind to amplification inhibitors present in the purification mixture;
(c) combining the purification mixture with a second set of PMPs having a surface material capable of non-specifically binding nucleic acids, and allowing nucleic acid from purification mixture to bind to the second set of PMPs;
(e) separating the second set of PMPs from a liquid fraction of the purification mixture; and
(f) contacting the second set of PMPs with an elution solution of 500 μl or less and allowing the nucleic acid from the purification mixture to elute off the second set of PMPs and into the elution solution.

44. A nucleic acid capture composition comprising:
(a) paramagnetic particles (PMPs) having a surface material capable of non-specifically binding nucleic acids, cells, and viral particles;
(b) divalent metal ions; and
(c) a surfactant.

45. The composition of claim 44, wherein the surface material comprises silica, cellulose, chitosan, agarose, or Sepharose.

46. The composition of claim 44, wherein the divalent metal ions are selected from barium (Ba²⁺), copper [II] (Cu²⁺), calcium (Ca²⁺), magnesium (Mg²⁺), manganese [II] (Mn²⁺), zinc (Zn²⁺), iron [II] (Fe²⁺), nickel (Ni²⁺), cobalt (Co²⁺), tin [II] (Sn²⁺), cadmium (Cd²⁺), and lead [II] (Pb²⁺).

47. The composition of claim 46, wherein the divalent metal ions are selected from Cut, Zn²⁺, Co²⁺, Fe²⁺, and/or Ni²⁺.

48. The composition of claim 47, wherein divalent metal ions comprise Zn²⁺.

49. The composition of claim 44, wherein the surfactant is a cationic surfactant.

50. The composition of claim 49, wherein the cationic surfactant is selected from behentrimonium chloride, benzalkonium chloride, benzethonium chloride, benzododecinium bromide, carbethopendecinium bromide, cetalkonium chloride, cetrimide, cetrimonium bromide, cetrimonium chloride, cetylpyridinium chloride, didecyldimethylammonium chloride, dimethyldioctadecylammonium bromide, dimethyldioctadecylammonium chloride, dioleoyl-3-trimethylammonium propane, domiphen bromide, lauryl methyl gluceth-10 hydroxypropyl dimonium chloride, octenidine dihydrochloride, olaflur, n-oleyl-1,3-propanediamine, pahutoxin, stearalkonium chloride, tetramethylammonium hydroxide, and thonzonium bromide.

51. The composition of claim 44, wherein the surfactant is a nonionic surfactant.

52. The composition of claim 51, wherein the nonionic surfactant is selected from alkyl polyglycoside, Cetomacrogol 1000, cetostearyl alcohol, cetyl alcohol, cocamide DEA, cocamide MEA, decyl glucoside, decyl polyglucose, glycerol monostearate, IGEPAL CA-630, isoceteth-20, Lauryl glucoside, Maltoside, monolaurin, mycosubtilin, narrow-range ethoxylate, nonidet P-40, nonoxynol-9, nonoxynols, NP-40, octaethylene glycol monododecyl ether, N-Octyl beta-D-thioglucopyranoside, octyl glucoside, oleyl alcohol, pentaethylene glycol monododecyl ether, polidocanol, Poloxamer, Poloxamer 407, polyethoxylated tallow amine, polyglycerol polyricinoleate, Polysorbate, Polysorbate 20, Polysorbate 80, Sorbitan, Sorbitan monolaurate, Sorbitan monostearate, Sorbitan tristearate, stearyl alcohol, Surfactin, Triton X-100, and Tween 80.

53. The composition of claim 44, wherein components (a)-(c) are provided in a liquid suspension.

54. A kit comprising a container capable of securely containing a large-volume sample and a composition of one of claims 44-53.

55. The composition of claim 44, wherein components (a)-(c) are provided as a dry reagent.

56. The composition of claim 55, wherein components (a)-(c) are provided within a sealed capsule, wherein the capsule comprises a material that will dissolve when added to a liquid sample.

57. The composition of claim 55, wherein components (a)-(c) are provided as a lyophilized powder, pellet, tablet, or disc.

58. A kit comprising a container having the composition of claim 57 contained therein and capable of securely containing a large-volume sample.

59. A method comprising combining a large-volume sample with a composition of one of claim 44-53 or 55-57.

60. A method comprising: (a) providing the kit of claim 54, and (b) combining a large-volume sample and the composition of one of claims 44-53 within the container.

61. A method comprising placing a large-volume sample within the container of the kit of claim 58.

62. The method of claim 59 or 60, wherein the large-volume sample is an environmental sample.

63. The method of claim 59 or 60, wherein the large-volume sample is an biological sample.

64. A composition comprising a pyrrolidone derivative and isopropanol.

65. The composition of claim 64, wherein the pyrrolidone derivative is polyvinylpolypyrrolidone (PVPP).

66. The composition of claim 65, wherein the pyrrolidone derivative is PVPP-co-polystyrene.

67. A method of removing or sequestering amplification inhibitors in a sample comprising contacting the sample with a composition of one of claims 64-66 and allowing the pyrrolidone derivative to bind to the amplification inhibitors.

68. The method of claim 67, further comprising removing the pyrrolidone-derivative-bound amplification inhibitors from the sample.

69. A method of preparing a large-volume sample for nucleic acid concentration comprising combining the large-volume sample with a first solid surface in the presence of a binding reagent, the binding reagent comprising divalent metal ions and a surfactant, and allowing the nucleic acids, cells, and/or viral particles in the large-volume sample to bind to the solid surface.

70. The method of claim 69, wherein the large-volume sample is a biological and/or environmental sample of 5 ml volume or greater.

71. The method of claim 69, wherein the large-volume sample contains free nucleic acids, nucleic acid complexes with other biological macromolecules, cells, and/or viral particles.

72. The method of claim 69, wherein the first solid surface is a well, tube, bead, chip, plate, particle, membrane, or filter.

73. The method of claim 72, wherein the first solid surface is a paramagnetic particle (PMP).

74. The method of claim 73, wherein the PMP comprises a surface material capable of non-specifically binding to nucleic acids.

75. The method of claim 74, wherein the surface material comprises silica, cellulose, chitosan, agarose, or Sepharose.

76. The method of claim 69, wherein the binding reagent comprises divalent metal ions selected from barium (Ba²⁺), copper [II] (Cu²⁺), calcium (Ca²⁺), magnesium (Mg²⁺), manganese [II] (Mn²⁺), zinc (Zn²⁺), iron [II] (Fe²⁺), nickel (Ni²⁺), cobalt (Co²⁺), tin [II] (Sn²⁺), cadmium (Cd²⁺), and lead [II] (Pb²⁺).

77. The method of claim 76, wherein the binding reagent comprises Cu+, Zn²⁺, Co²⁺, Fe²⁺, and/or Ni²⁺.

78. The method of claim 77, wherein the binding reagent comprises Zn²⁺.

79. The method of claim 69, wherein the binding reagent comprises a cationic surfactant.

80. The method of claim 79, wherein the cationic surfactant is selected from behentrimonium chloride, benzalkonium chloride, benzethonium chloride, benzododecinium bromide, carbethopendecinium bromide, cetalkonium chloride, cetrimide, cetrimonium bromide, cetrimonium chloride, cetylpyridinium chloride, didecyldimethylammonium chloride, dimethyldioctadecylammonium bromide, dimethyldioctadecylammonium chloride, dioleoyl-3-trimethylammonium propane, domiphen bromide, lauryl methyl gluceth-10 hydroxypropyl dimonium chloride, octenidine dihydrochloride, olaflur, n-oleyl-1,3-propanediamine, pahutoxin, stearalkonium chloride, tetramethylammonium hydroxide, and thonzonium bromide.

81. The method of claim 69, wherein the binding reagent comprises a nonionic surfactant.

82. The method of claim 81, wherein the nonionic surfactant is selected from alkyl polyglycoside, Cetomacrogol 1000, cetostearyl alcohol, cetyl alcohol, cocamide DEA, cocamide MEA, decyl glucoside, decyl polyglucose, glycerol monostearate, IGEPAL CA-630, isoceteth-20, Lauryl glucoside, Maltoside, monolaurin, mycosubtilin, narrow-range ethoxylate, nonidet P-40, nonoxynol-9, nonoxynols, NP-40, octaethylene glycol monododecyl ether, N-Octyl beta-D-thioglucopyranoside, octyl glucoside, oleyl alcohol, pentaethylene glycol monododecyl ether, polidocanol, Poloxamer, Poloxamer 407, polyethoxylated tallow amine, polyglycerol polyricinoleate, Polysorbate, Polysorbate 20, Polysorbate 80, Sorbitan, Sorbitan monolaurate, Sorbitan monostearate, Sorbitan tristearate, stearyl alcohol, Surfactin, Triton X-100, and Tween 80.
